(19) United States
(12) Patent Application Publication
(10) Pub. No.: US 2023/0048607 A1
(43) Pub. Date: Feb. 16, 2023

(54) MEDICAL DEVICES, SYSTEMS, AND METHODS FOR MEASURING MUSCULOSKELETAL PARAMETERS

(71) Applicant: Orthosensor Inc., Dania Beach, FL (US)

(72) Inventors: Ryan Ortiz, Miramar, FL (US); Glenn Lewis Monroe, Boca Raton, FL (US); Bob Chilton, Dania Beach, FL (US); Andrew Chase, Dania Beach, FL (US); Min Sic Roh, Dania Beach, FL (US)

(73) Assignee: Orthosensor Inc., Dania Beach, FL (US)

(21) Appl. No.: 17/819,120

(22) Filed: Aug. 11, 2022

Related U.S. Application Data
(60) Provisional application No. 63/232,818, filed on Aug. 13, 2021.

(51) Int. Cl. A61F 2/46; A61F 2/48

(52) U.S. Cl. CPC A61F 2/4657; A61F 2/482; A61F 2002/4666

(57) ABSTRACT

A measurement device is disclosed that includes a first component having an outer surface having one or more flexible articular surfaces, and an inner surface having a first area having protrusions defining a polygon with a plurality of vertices. A load plate can be in contact with the first area. A printed circuit board can have a central section and a first lateral section. The first lateral section can have a sensor array having a plurality of sensors. Each sensor can be positioned in alignment with a vertex of the polygon and having a load pad in contact with a lower surface of the rigid load plate. A reference sensor can be spaced from the lower surface of the load plate.

### CROSS-REFERENCE TO RELATED APPLICATIONS

This patent claims the benefit of priority under 35 U.S.C. § 119 to U.S. Provisional Patent Application No. 63/232,818, filed Aug. 13, 2021, which is herein incorporated by reference in its entirety.

### FIELD

The present disclosure relates generally to measurement of physical parameters, and particularly to, but not exclusively, medical electronic devices for high precision sensing.

### BACKGROUND

The skeletal system of a mammal is subject to variations among species. Further changes can occur due to environmental factors, degradation through use, and aging. An orthopedic joint of the skeletal system typically includes two or more bones that move in relation to one another. Movement is enabled by muscle tissue and tendons attached to the skeletal system of the joint. Ligaments hold and stabilize the one or more joint bones positionally. Cartilage is a wear surface that prevents bone-to-bone contact, distributes load, and lowers friction.

There has been substantial growth in the repair of the human skeletal system. In general, orthopedic joints have evolved using information from simulations, mechanical prototypes, and patient data that is collected and used to initiate improved designs. Similarly, the tools being used for orthopedic surgery have been refined over the years but have not changed substantially. Thus, the basic procedure for replacement of an orthopedic joint has been standardized to meet the general needs of a wide distribution of the population.

Although the tools, procedure, and artificial joint meet a general need, each replacement procedure is subject to significant variation from patient to patient. The correction of these individual variations relies on the skill of the surgeon to adapt and fit the replacement joint using the available tools to the specific circumstance. The solutions of this disclosure resolves these and other issues of the art.

### SUMMARY

In accordance with certain embodiments of the present disclosure, a system for measuring one or more parameters of the muscular-skeletal system is disclosed. The system can include a first component with an outer surface having one or more flexible articular surfaces and an inner surface having a first area having protrusions defining a polygon with a plurality of vertices. A load plate can be in contact with the first area. A printed circuit board can include a central section and a first lateral section. The first lateral section can have a sensor array having a plurality of sensors, each sensor being positioned in alignment with a vertex of the polygon. A reference sensor can be paced from the lower surface of the load plate such that a load applied to the outer surface of the first component compresses at least one of the plurality of sensors while the reference sensor remains unloaded. Electronic circuitry can be included and be configured to control a measurement process, receive measurement data from the sensor array, and transmit measurement data. A second component can be configured to attach to the first component to form a hermetically sealed housing for the printed circuit board.

In some aspects, at least two of the plurality of sensors can be positioned aligned on opposite vertices of an inner lateral edge of the polygon. Each of the plurality of sensors may include a load pad in contact with a lower surface of the rigid load plate. Lateral section can have a curved outer edge opposite a relatively non-curved inner edge. The reference sensor of each lateral section can be positioned adjacent the relatively non-curved inner edge and between the sensors positioned on the opposite vertices.

In some aspects, a third sensor of the plurality of sensors is centrally positioned along a curved edge of the sensor array opposite a non-curved inner edge of the sensor array.

In some aspects, lateral section of the printed circuit board has one or more raised columns configured to aligned with sensors of sensor array.

In some aspects, the inner surface has a second area having at least one protrusion forming a polygon with a plurality of vertices and a second load plate is positioned in contact with the second area. The printed circuit board has a second lateral section opposite the first lateral section and a second sensor array having a plurality of sensors aligned with a vertex of the polygon of the second area, and in some examples having a load pad in contact with a lower surface of the second rigid load plate. A second reference sensor can be spaced from the lower surface of the second load plate such that a load applied to the outer surface of the first component compresses at least one of the plurality of sensors while the reference sensor remains unloaded.

In some aspects, traces of the printed circuit board electrically couple the reference sensor and each of the plurality of sensors of the sensor array, and each trace may include at least one coil or dense portion.

In some aspects, traces of the printed circuit board that electrically couple the reference sensor and each of the plurality of sensors of the sensor array may each have an approximately equivalent length, and each trace may be configured to have an equal base capacitance (e.g., approximately 16.4 pF).

In some aspects, traces of the printed circuit board that electrically couple the reference sensor and each of the plurality of sensors of the sensor array may include dense portions, and each dense portion may be spaced from each other.

In some aspects, the reference sensor is configured to be used for calibration.

In some aspects, each of the sensors may include a compressible capacitor with an elastic dielectric. At least one of the plurality of sensors can have alternating conductive and dielectric layers forming at least one capacitor where deformation of at least one of the dielectric layers in response to musculoskeletal loading changes a capacitance of the capacitor. In some examples, each of the sensors may include a plurality of capacitors.

In some aspects, the at least one capacitor can have more than one capacitor mechanically in series.

In some aspects, a measurement device is disclosed that includes a first component having an outer surface having one or more articular surfaces and an inner surface having an area having at least one protrusion defining a polygon. A load plate can be in contact with the at least one protrusion. A printed circuit board can be included having a central section and a lateral section extended from the central section. The lateral section can have a sensor array having a plurality of sensors, and each of the plurality of sensors may be in contact with a lower surface of the load plate. In some examples, each of the plurality of sensors may include a load pad etched down and in contact with a lower surface of the load plate. The sensor array can have a first sensor positioned in a central portion of a periphery of the lateral section, a second sensor in a first corner of the periphery, and a third sensor in a second corner opposite the first corner of periphery, each of the first, second, and third sensors being aligned with vertices of the polygon. A reference sensor can be positioned between the second and third sensors offset from an inner non-curved edge of the periphery and spaced from the lower surface of the load plate. Electronic circuitry can be configured to control a measurement process, receive measurement data, and transmit data from the plurality of sensors and the reference sensor. A second component may be configured to attach to the first component to form a housing for the printed circuit board.

In some examples, each of the sensors can have alternating conductive and dielectric layers forming at least one capacitor where deformation of at least one of the dielectric layers in response to load changes a capacitance of the capacitor. Layers of the capacitor can be configured to resist compression in a spring-like manner.

In some examples, the outer surface of the first component has a material thickness relatively thin so that musculoskeletal loading is transferred from at least one of the areas having the inwardly oriented protrusions and at least one of the rigid load plates.

In some examples, the first component further may include a bias selectively positioned so that musculoskeletal loading that sit too mesial is transferred towards a lowest spot of the outer surface of the first component.

In some examples, the polygon is triangular shaped with an inner lateral edge aligned parallel with an inner lateral edge of the load plate, at least two of the plurality of sensors positioned on opposite vertices of the inner lateral edge.

In some examples, a method of assembling a measurement device is disclosed. The method can include attaching a printed circuit board to an inner surface of a base component of the measurement device, the printed circuit board having a central section and a lateral section, the lateral section having a sensor array having a plurality of sensors; positioning a lower surface of a load plate abutting an upper surface of the plurality of sensors of the sensor array, wherein the reference sensor is spaced from the lower surface of the load plate; and attaching an upper component to the lower component of the measurement device to form a hermetically sealed housing for the printed circuit board and the load plate, the upper component having an outer surface having one or more flexible articular surfaces and an inner surface having at least one protrusion forming a polygon with a plurality of vertices, wherein each vertex of the plurality of vertices is aligned with a sensor of the sensor array.

In some aspects, the method includes applying load to the outer surface of the upper component to compresses at least one of the plurality of sensors while the reference sensor remains unloaded.

In some aspects, the method includes positioning on or more raised columns of the printed circuit board in alignment with each sensor of the sensor array.

In some aspects, the method includes electrically coupling the central section of the printed circuit board to the reference sensor and each of the plurality of sensors of the sensor array with a corresponding trace. In some examples, each of the traces may include at least one coil or dense portion. In some examples, each of the traces may be substantially equal in length.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE FIGURES

The accompanying drawings, which are incorporated in and constitute a part of this specification, illustrate various exemplary aspects of the disclosure, and together with the description serve to explain the principles of the present disclosure.

FIG. 1 illustrates a measurement device positioned between a femur and a tibia and a receiving station, in accordance with an example embodiment.

FIG. 2A illustrates an upper perspective view of a measurement device, in accordance with an example embodiment.

FIG. 2B illustrates a lower perspective view of the measurement device of FIG. 2A.

FIG. 2C illustrates an exploded view of the measurement device of FIG. 2A.

FIG. 3A illustrates an upper perspective view of the measurement device of FIG. 2A with certain components removed for illustrative purposes.

FIG. 3B illustrates a top view of the measurement device of FIG. 2A with certain components removed for illustrative purposes.

FIG. 4A illustrates a magnified, partial cross-sectional view of section 4A-4A of FIG. 3B.

FIG. 4B illustrates a magnified, partial cross-sectional view of section 4B-4B of FIG. 3B.

FIG. 5A illustrates a top view of an example upper support structure of the measurement device of FIG. 2A.

FIG. 5B illustrates a bottom view of the example upper support structure of FIG. 5A.

FIG. 5C illustrates a magnified lower perspective view of a portion of the example upper support structure of FIG. 5A.

FIG. 6A illustrates an upper perspective view of a sensor array in accordance with an example embodiment.

FIG. 6B illustrates a top schematic view of the sensor array of FIG. 6A.

FIG. 7 illustrates a partial cross-sectional view of capacitors, in accordance with an example embodiment.

FIG. 8 depicts a flow diagram of a method of assembling a measurement device according to aspects of this disclosure.

### DETAILED DESCRIPTION

Particular aspects of the present disclosure are described in greater detail below. The terms and definitions provided herein control, if in conflict with terms and/or definitions incorporated by reference.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise.

The terms “approximately” and “about” refer to being nearly the same as a referenced number or value. As used herein, the terms “approximately” and “about” should be understood to encompass ±10% of a specified amount or value (e.g., “about 90%” can refer to the range of values from 81% to 99%.

As discussed herein, “operator” can include a doctor, surgeon, other health care professional, or any other individual or delivery instrumentation associated with operation of one or more measurement devices and related systems, as described throughout this disclosure.

Embodiments of the disclosure are broadly directed to measurement of physical parameters, and more particularly, to measurement of one or more parameters of a joint.

The following description of exemplary embodiment(s) is merely illustrative in nature and is in no way intended to limit the invention, its application, or uses.

For simplicity and clarity of the illustration(s), elements in the figures are not necessarily to scale, are only schematic, are non-limiting, and the same reference numbers in different figures denote the same elements, unless stated otherwise. Additionally, descriptions and details of well-known steps and elements are omitted for simplicity of the description. Notice that once an item is defined in one figure, it may not be discussed or further defined in the following figures.

The terms “first”, “second”, “third” and the like in the Claims or/and in the Detailed Description are used for distinguishing between similar elements and not necessarily for describing a sequence, either temporally, spatially, in ranking or in any other manner. It is to be understood that the terms so used are interchangeable under appropriate circumstances and that the embodiments described herein are capable of operation in other sequences than described or illustrated herein.

Processes, techniques, apparatus, and materials as known by one of ordinary skill in the art may not be discussed in detail but are intended to be part of the enabling description where appropriate.

The orientation of the x, y, and z-axes of rectangular Cartesian coordinates is assumed to be such that the x and y axes define a plane at a given location, and the z-axis is normal to the x-y plane. The axes of rotations about the Cartesian axes of the device are defined as yaw, pitch and roll. With the orientation of the Cartesian coordinates defined in this paragraph, the yaw axis of rotation is the z-axis through body of the device. Pitch changes the orientation of a longitudinal axis of the device. Roll is rotation about the longitudinal axis of the device.

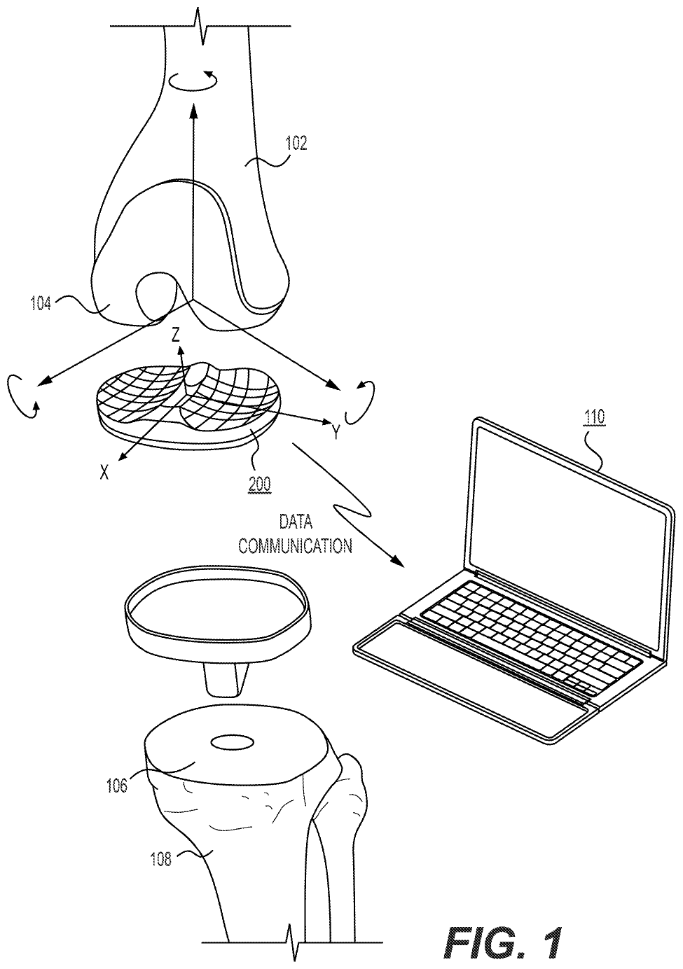

The orientation of the X, Y, Z axes (e.g., see FIG. 1) of rectangular Cartesian coordinates is selected to facilitate graphical display on computer screens having the orientation that the user will be able to relate to most easily. Therefore the image of the device moves upward on the computer display whenever the device itself moves upward for example away from the surface of the earth. The same applies to movements to the left or right.

Although inertial sensors are provided as enabling examples in the description of embodiments, any tracking device (e.g., a GPS chip, acoustical ranging, accelerometer, magnetometer, gyroscope, inclinometers, MEMs devices) can be used within the scope of the embodiments described.

At least one embodiment is directed to a kinetic orthopedic measurement system to aid a surgeon in determining real-time alignment, range of motion, loading, impingement, and contact point of orthopedic implants under load. In some examples, measurement device discussed herein may be used as a trialing implant, a permanent implant, or a tool. The measurement device may be used in the spine, shoulder, knee, hip, ankle, wrist, finger, toe, or other portions of a musculoskeletal system. In a non-limiting example disclosed herein a medical system including a measurement device is illustrated to support the implantation of a knee joint.

The non-limiting embodiment described herein is referred to herein as the kinetic system. The kinetic system includes a sensor system that provides quantitative measurement data and feedback that can be provided visually, audibly, or haptically to a surgeon, surgical team, and/or surgical robotic system. The kinetic system provides the surgeon real-time dynamic data regarding force, pressure, or loading within the musculoskeletal system, contact and congruency through a full range of motion, and information regarding impingement, among other parameters.

In general, kinetics is the study of the effect of forces upon the motion of a body or system of bodies. Disclosed herein is a system for kinetic assessment of the musculoskeletal system. The kinetic system can be for general measurement of the musculoskeletal system, trial installation and measurement of prosthetic components, or long-term monitoring of an installed permanent prosthetic component to the musculoskeletal system. For example, in an installation of a trialing prosthetic component one or more bone surfaces have to be prepared to receive a device or prosthetic component.

The kinetic system is designed to take quantitative measurements of at least the load, position of load, or alignment with the forces being applied to the joint similar to that of a final joint installation. The kinetic system can support the actual bone cut for optimal contact point(s), balance, load magnitude, and alignment over a range of motion. The one or more measurement components having sensors are designed to allow ligaments, tissue, and bone to be in place while the quantitative measurement data is taken and reported in real-time. This is significant because the bone cuts may be adjusted to take into account the kinetic forces from the kinematic assessment, and subsequent adjustments of the bone cuts may change the alignment, load, and position of load once the joint is reassembled. Furthermore, the measurement data can be transmitted to a computer in the operating room that can analyze the measurement data and propose a workflow for the surgical team to yield the desired results. Moreover, the kinetic system supports real-time adjustments, such as bone cuts, rotation of a prosthetic component, or ligament tensioning, with real-time measurements to validate the surgical procedure or the proposed workflow.

A prosthetic joint installation can benefit from quantitative measurement data in conjunction with subjective feedback of the prosthetic joint to the surgeon. The quantitative measurements can be used to determine adjustments to bone, prosthetic components, or tissue prior to final installation. Permanent sensors can also be housed in final prosthetic components to provide periodic data related to the status of the implant, such as implant positioning or joint range of motion. Data collected intra-operatively and post-operatively (long-term) can be used to determine parameter ranges for surgical installation and to improve future prosthetic components.

The physical parameter or parameters of interest can include, but are not limited to, measurement of alignment, load, force, pressure, position, displacement, density, viscosity, pH, spurious accelerations, color, movement, particulate matter, structural integrity, and localized temperature. Often, several measured parameters are used to make a quantitative assessment. A graphical user interface can support assimilation of measurement data. Parameters can be evaluated relative to orientation, alignment, direction, displacement, or position as well as movement, rotation, or acceleration along an axis or combination of axes by wireless sensing modules or devices positioned on or within a body, instrument, appliance, vehicle, equipment, or other physical system.

While the specification concludes with claims defining the features of the invention that are regarded as novel, it is believed that the invention will be better understood from a consideration of the following description in conjunction with the drawing figures, in which like reference numerals are carried forward.

The example embodiments shown herein below of the measurement device are illustrative only and does not limit use for other parts of a body. The measurement device can be a tool, equipment, implant, or prosthesis that measures at least one parameter or supports installation of prosthetic components to the musculoskeletal system. The measurement device can be used on bone, the knee, hip, ankle, spine, shoulder, hand, wrist, foot, fingers, toes, and other areas of the musculoskeletal system. In general, the principles disclosed herein are meant to be adapted for use in all locations of the musculoskeletal system.

Processes, techniques, apparatus, and materials as known by one of ordinary skill in the art may not be discussed in detail but are intended to be part of the enabling description where appropriate. For example, specific computer code may not be listed for achieving each of the steps discussed, however one of ordinary skill would be able, without undo experimentation, to write such code given the enabling disclosure herein. Such code is intended to fall within the scope of at least one exemplary embodiment.

In all of the examples illustrated and discussed herein, any specific materials, such as temperatures, times, energies, and material properties for process steps or specific structure implementations should be interpreted to be illustrative only and non-limiting. Processes, techniques, apparatus, and materials as known by one of ordinary skill in the art may not be discussed in detail but are intended to be part of an enabling description where appropriate. It should also be noted that the word “coupled” used herein implies that elements may be directly coupled together or may be coupled through one or more intervening elements.

Additionally, the sizes of structures used in exemplary embodiments are not limited by any discussion herein (e.g., the sizes of structures can be macro (centimeter, meter, and larger sizes), micro (micrometer), and nanometer size and smaller).

Notice that similar reference numerals and letters refer to similar items in the following figures, and thus once an item is defined in one figure, it may not be discussed or further defined in the following figures.

FIG. 1 is an illustration of a measurement device 200 positioned between a femur 102 and a tibia 108 for measuring one or more parameters in accordance with an exemplary embodiment. In general, device 200 is placed in contact with or in proximity to the muscular-skeletal system to measure a parameter. In a non-limiting example, device 200 is used to measure a parameter of a muscular-skeletal system during a procedure, such as an installation of an artificial joint.

Embodiments of device 200 are broadly directed to measurement of physical parameters, and more particularly, to evaluating changes in the transit time of a pulsed energy wave propagating through a medium. In-situ measurements during orthopedic joint implant surgery would be of substantial benefit to verify an implant is in balance and under appropriate loading or tension. In one embodiment, the instrument is similar to and operates familiarly with other instruments currently used by surgeons. This will increase acceptance and reduce the adoption cycle for a new technology. The measurements will allow the surgeon to ensure that the implanted components are installed within predetermined ranges that maximize the working life of the joint prosthesis and reduce costly revisions. Providing quantitative measurement and assessment of the procedure using real-time data will produce results that are more consistent. A further issue is that there is little or no implant data generated from the implant surgery, post-operatively. Device 200 can provide implant status data to the orthopedic manufacturers and surgeons. Moreover, data generated by direct measurement of the implanted joint itself would greatly improve the knowledge of implanted joint operation and joint wear thereby leading to improved design and materials. In at least one exemplary embodiment, an energy pulse is directed within one or more waveguides in device 200 by way of pulse mode operations and pulse shaping. The waveguide is a conduit that directs the energy pulse in a predetermined direction. The energy pulse is typically confined within the waveguide. In one embodiment, the waveguide includes a polymer material.

For example, urethane or polyethylene are polymers suitable for forming a waveguide. The polymer waveguide can be compressed and has little or no hysteresis in the system. Alternatively, the energy pulse can be directed through the muscular-skeletal system. In one embodiment, the energy pulse is directed through bone of the muscular-skeletal system to measure bone density. A transit time of an energy pulse is related to the material properties of a medium through which it traverses. This relationship is used to generate accurate measurements of parameters such as distance, weight, strain, pressure, wear, vibration, viscosity, and density to name but a few.

Device 200 can be size constrained by form factor requirements of fitting within a region the muscular-skeletal system or a component such as a tool, equipment, or artificial joint. In a non-limiting example, device 200 is used to measure load and balance of an installed artificial knee joint. A knee prosthesis includes a femoral prosthetic component 104, an insert, and a tibial prosthetic component 106, as shown in FIG. 1. A distal end of femur 102 is prepared and receives femoral prosthetic component 104. Femoral prosthetic component 104 typically has two condyle surfaces that mimic a natural femur. Femoral prosthetic component 104 can have a single condyle surface coupled to femur 102. Femoral prosthetic component 104 is typically made of a metal or metal alloy.

A proximal end of tibia 108 is prepared to receive tibial prosthetic component 106. Tibial prosthetic component 106 is a support structure that is fastened to the proximal end of the tibia and is usually made of a metal or metal alloy. The tibial prosthetic component 106 also retains the insert in a fixed position with respect to tibia 108. The insert is fitted between femoral prosthetic component 104 and tibial prosthetic component 106. The insert has at least one bearing surface that is in contact with at least condyle surface of femoral prosthetic component 104. The condyle surface can move in relation to the bearing surface of the insert such that the lower leg can rotate under load. The insert is typically made of a high wear plastic material that minimizes friction.

In a knee joint replacement process, the surgeon affixes femoral prosthetic component 104 to the femur 102 and tibial prosthetic component 106 to tibia 108. The tibial prosthetic component 106 can include a tray or plate affixed to the proximal end of the tibia 108. Device 200 is placed between a condyle surface of femoral prosthetic component 104 and a major surface of tibial prosthetic component 106. The condyle surface contacts a major surface of device 200. The major surface of device 200 approximates a surface of the insert. Tibial prosthetic component 106 can include a cavity or tray on the major surface that receives and retains device 200 during a measurement process. Tibial prosthetic component 106 and device 200 has a combined thickness that represents a combined thickness of tibial prosthetic component 106 and a final (or chronic) insert of the knee joint.

In one embodiment, device 200 can include sensors, whereby each sensor can be independent and measure information. Electronics can be shared between the sensors to lower cost and complexity of the system. The shared electronics can multiplex between each sensor module to take measurements when appropriate. Measurements taken by device 200 aid the surgeon in modifying the absolute loading on each condyle and the balance between condyles. Although shown for a knee implant, device 200 can be used to measure other orthopedic joints such as the spine, hip, shoulder, elbow, ankle, wrist, interphalangeal joint, metatarsophalangeal joint, metacarpophalangeal joints, and others.

Alternatively, device 200 can also be adapted to orthopedic tools to provide measurements. The prosthesis incorporating device 200 can emulate the function of a natural knee joint. Device 200 can measure loads or other parameters at various points throughout the range of motion. Data from device 200 is transmitted to a receiving station 110 via wired or wireless communications. In one embodiment, device 200 is a disposable system. Device 200 can be disposed of after using device 200 to optimally fit the joint implant. Device 200 may be a low cost disposable system that reduces capital costs, operating costs, facilitates rapid adoption of quantitative measurement, and initiates evidentiary based orthopedic medicine.

In another embodiment, a methodology can be put in place to clean and sterilize device 200 for reuse. In another embodiment, device 200 can be incorporated in a tool instead of being a component of the replacement joint. The tool can be disposable or be cleaned and sterilized for reuse. In another embodiment, device 200 can be a permanent component of the replacement joint. Device 200 can be used to provide both short term and long term post-operative data on the implanted joint. Device 200 may be be coupled to the muscular-skeletal system. In any of the embodiments, receiving station 110 can include data processing components, electronic storage components, and/or display components, and may provide real time graphical representation of the level and distribution of a load. Receiving station 110 can record measured parameters and provide accounting information of device 200 to an appropriate authority.

In an intra-operative example, device 200 can measure forces (Fx, Fy, Fz) with corresponding locations and torques (e.g. Tx, Ty, and Tz) on the femoral prosthetic component 104 and the tibial prosthetic component 106. The measured force and torque data is transmitted to receiving station 110 to provide real-time visualization for assisting the surgeon in identifying any adjustments needed to achieve optimal joint pressure and balancing. The data has substantial value in determining ranges of load and alignment tolerances required to minimize rework and maximize patient function and longevity of the joint.

As mentioned previously, device 200 can be used for other joint surgeries, meaning, device 200 is not limited to total or partial knee replacement procedures. Moreover, device 200 is not limited to trial measurements. Device 200 can be incorporated into the final joint system to provide data post-operatively to determine if the implanted joint is functioning correctly. Early determination of a problem using device 200 can reduce failure of the joint by bringing awareness to a problem that the patient cannot detect. The problem can often be rectified with a minimal invasive procedure at lower cost and stress to the patient. Similarly, longer term monitoring of the joint can determine wear or misalignment that if detected early can be adjusted for optimal life or replacement of a wear surface with minimal surgery thereby extending the life of the implant. In general, device 200 can be shaped such that it can be placed or engaged or affixed to or within load bearing surfaces used in many orthopedic applications (or used in any orthopedic application) related to the musculoskeletal system, joints, and tools associated therewith. Device 200 can provide information on a combination of one or more performance parameters of interest such as wear, stress, kinematics, kinetics, fixation strength, ligament balance, anatomical fit, and joint balance.

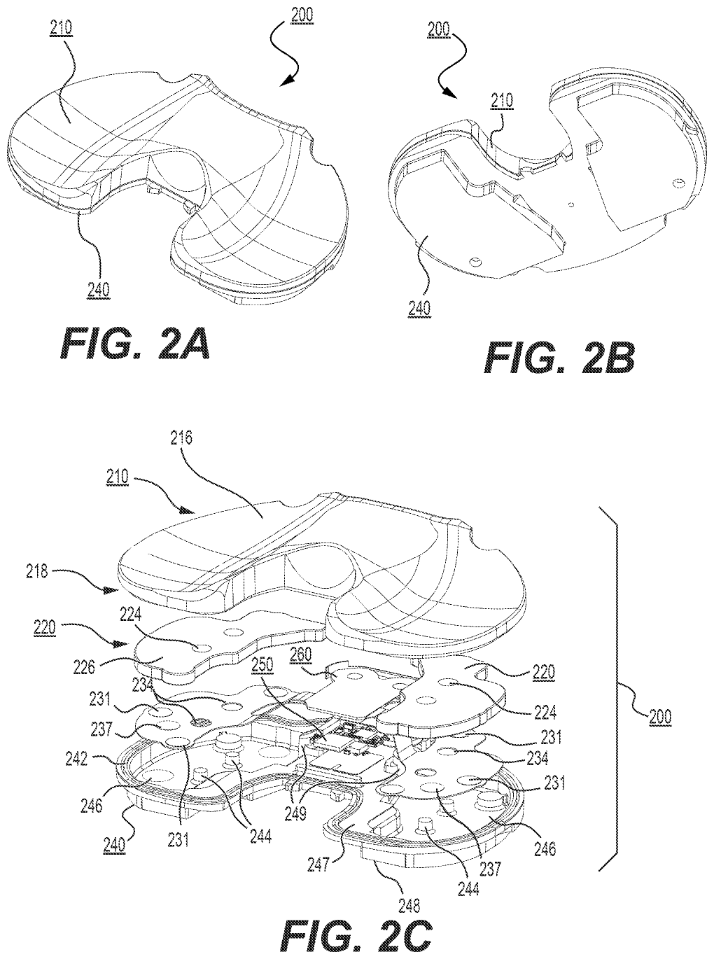

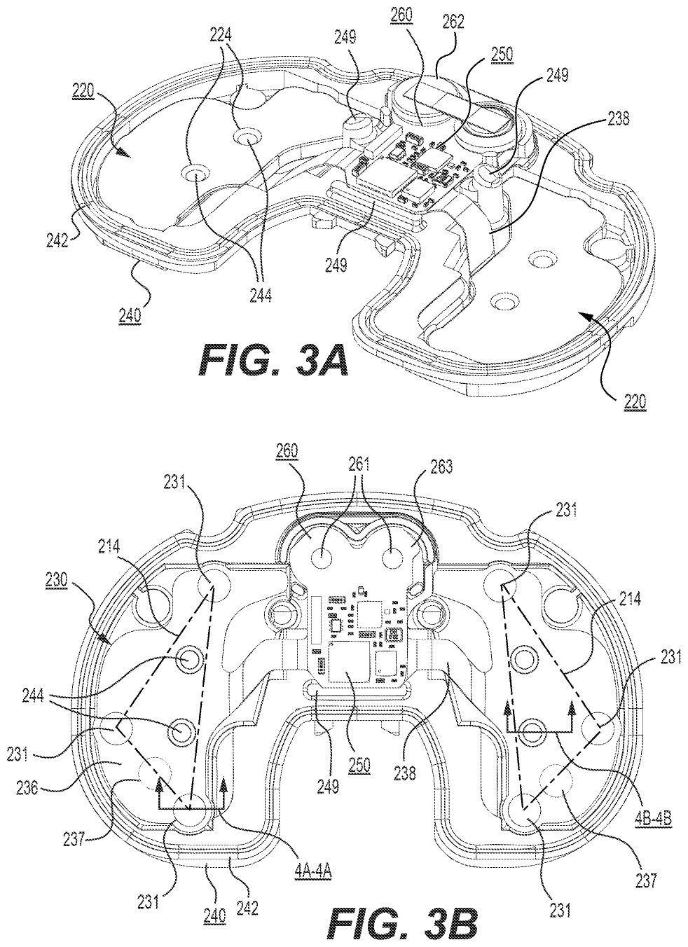

Referring now to FIGS. 2A-2C, example measurement device 200 is illustrated. Measurement device 200 can measure a force, pressure, or load, and the position of the force, pressure, or load, on a surface. As illustrated, device 200 can include or be integrated with equipment, tools, prosthetic components, or devices that couple to the muscular-skeletal system. Device 200 can include any of the packaging, circuits, power sources, sensors, system architecture, remote systems, or application integrated circuits, disclosed herein or incorporated by reference. In particular, FIG. 2A illustrates an upper perspective view of measurement device 200 in an assembled state, while FIG. 2B illustrates a lower perspective view of device 200 in the assembled state. FIG. 2C shows measurement device 200 in an exploded state with an upper support structure 210 capable of coupling to a lower support structure 240 to form an enclosure or housing of device 200. FIG. 2C shows electronic circuitry 250 below section 263 of printed circuit board 260 strictly for illustration purposes. When assembled and not the exploded state of FIG. 2C, it can be seen in FIG. 3A that electronic circuitry 250 is mounted to printed circuit board 260. Within the enclosure formed by structures 210, 240, measurement device 200 can include load plates 220, printed circuit board 260, electronic circuitry 250, and a pair of sensor arrays 230. In one embodiment, three sensors can be included in sensor array 230 and device 200 can include a sensor array 230 for each knee compartment. Each sensor array 230 can be used to measure information, such as the load and position of load of a knee compartment. Electronic circuitry 250 couples to and controls measurement from sensor arrays 230.

Support structures 210, 240 can couple to femoral prosthetic component 104 and tibial prosthetic component 106, as in FIG. 1. Although shown as an insert for the knee, measurement device 200 is suitable for integration into prosthetic components for the hip, spine, shoulder, ankle, elbow, bone, hand, and feet. As mentioned previously, measurement device 200 can be integrated into tools or equipment that couple to the muscular-skeletal system for providing force, pressure, or load information such as a distractor, cutting jig, spacer, orthopedic screw, robot, or other tool. Support structure 210 can include one or more surfaces that couples to the tibial prosthetic component 106, such as articular surfaces 216 for coupling to and allowing articulation therewith. The outer, articulated surfaces 216 can be configured to be shaped to accommodate corresponding condyle surfaces of a femoral insert.

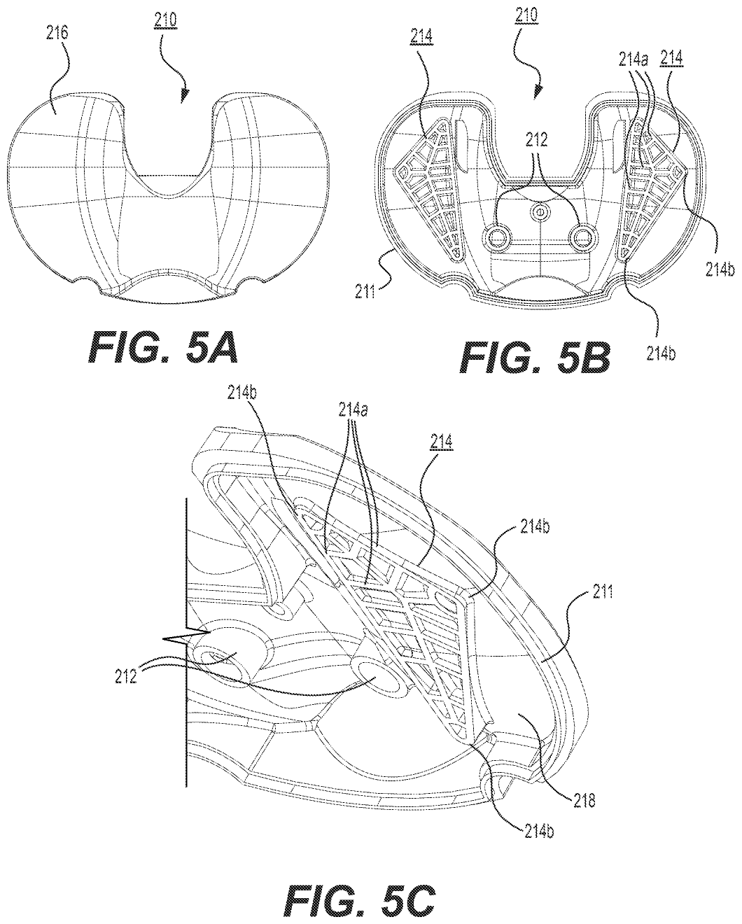

As shown in FIG. 2C and more particularly in FIGS. 5A-5C, an underside 218 of support structure 210 has a lower load support area 214. Lower load support area 214 may be defined by protrusions 214a protruding from the underside 218 of support structure 210, and vertices 214b of protrusions 214a may form a polygon. In the example, the polygon forming area 214 is a triangle with a matrix of ribbed protrusions 214a extended inward away from the underside 218 of support structure 210. While a triangle is shown, area 214 can be any shape. Area 214, which is shown in FIG. 3B in dashed lines, can relate or otherwise correspond to sensors of sensor array 230 and reference sensor 237 of sensor array 230 positioned therebelow (e.g., at vertices 214b of area 214, along a perimetral edge of area 214, etc.). Area 214 is configured to align to load plates 220, and protrusions 214a may couple to load plates 220.

Structure 210 can also include alignment features 212 (shown in FIG. 5B), which are configured to be inserted over one or more alignment features 249 of support structure 240 when support structures 210, 240 are coupled together in the assembled state of FIGS. 2A and 2B. Alignment features 212, 249 can be used to precisely align sensor arrays 230 to support structure 210, including to area 214 and corresponding articular surfaces 218. This is particularly advantageous as it allows the position of the sensors of sensor array 230 and/or reference sensor 237 in relation to articular surfaces 218 to be used in the calculation to determine where a force, pressure, or load is applied to articular surfaces 218.

Support structure 210 can include a peripheral flange 211 that couples with a corresponding peripheral flange 242 of support structure 240. In one embodiment, flange 211 of support structure 210 can be inserted into a receiving channel of flange 242 and bonded thereto by an adhesive or glue. Flange 211 can also be a barrier to the ingress or egress of foreign solids, liquids, or gas into and out of measurement device 200. Flange 211 can also provide a relatively large bonding area for the adhesive to ensure a hermetic seal of measurement device 200.

In some aspects, a load path between surfaces 216, area 214, and sensors of sensor array 230 can be configured to divert load into the sensor array 230 in each condyle. In some aspects, the relative thinness of structure 210, including at surfaces 216, can allow for load transfer to occur from area 214 to plate 220 therebelow. In some aspects, structure 210 being relatively thin around area 214 allows for most of the load path energy to pass through area 214.

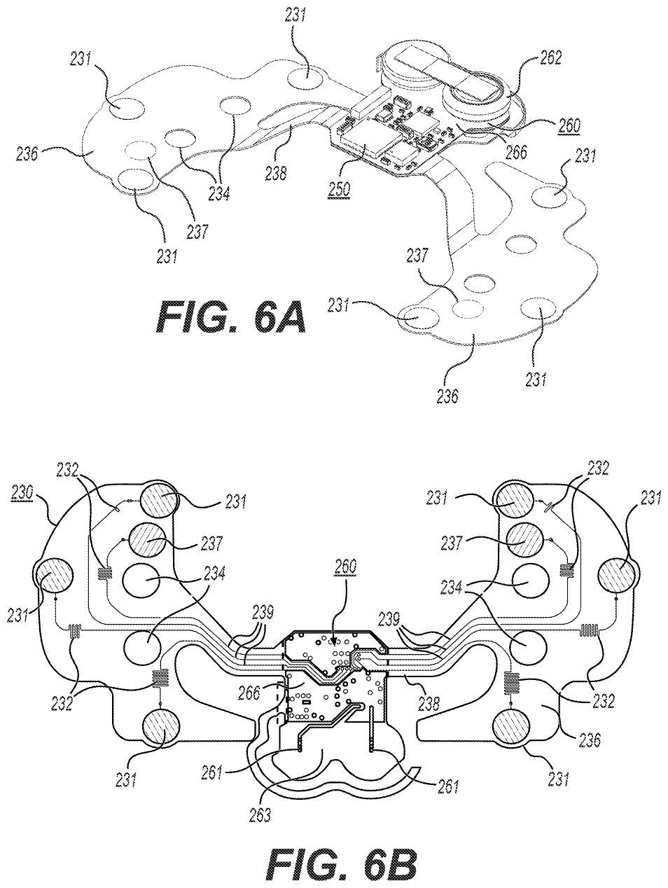

Referring now to printed circuit board 260 as shown in FIG. 6B, electronic circuitry 250 can be mounted on and coupled by metal traces 239 formed on printed circuit board 260. In some aspects, printed circuit board 260 can include one or more layers with capacitors built into it, whereby material selection can create the layers of the capacitors. In some aspects, the materials of printed circuit board 260 can be configured to equate the compression due to a force to a spring under compression. The force can be the load range in which the device will be used. In turn, the materials of printed circuit board 260 can be tailored to the load range that device 200 will be used in so that sensors of sensor arrays 230 deflect in a manner that is preferable for the rest of device 200. The plastics and the metals of printed circuit board 260 can be configured to deflect a certain amount with the loads that device 200 undergoes. Printed circuit board 260 can be configured to operate within those specific ranges so that all materials are within respective yield curves and therefore will spring back to original shape and position.

In the examples shown in FIGS. 6A and 6B, printed circuit board 260 is shown including at least three sections. A first section 263 of printed circuit board 260 can be located centrally between lateral sections 236. Electronic circuitry 250 can be located on section 263. Sections 236 of printed circuit board 260 extend laterally via bridge 238 from section 263. The sections 236 and bridge 238 can be integrally formed with each other and include alignment features 234. In some aspects, sections 236 can be a pair of substantially hemispherical shape substrates interconnected with section 263 via bridge 238. In some examples, sections 236 can include a curved outer edge 236a opposite a relatively straight, non-curved inner edge 236b with at least two pads 231 positioned aligned on opposite vertices of the inner lateral edge of array 230. While each of sections 236 is depicted with a hemispherical shape, other shapes are contemplated within the scope of this disclosure, including but not limited to circular, elliptical, oblong, polygonal, or any other shape, as needed or required. Sections 236 can couple sensors of sensor array 230 and reference sensor 237 to electronic circuitry 250.

In one example, a sensor of sensor array 230 can be positioned in opposite vertices or corner of array 230, as shown where pads 231 are positioned, while another sensor of array 230 can be centrally positioned along a periphery of array 230 between the pair of opposite corner vertices. Load pads 231 can underlie and support load plates 220 for directing a force, pressure, or load applied to articular surfaces 216 to the sensors of array 230.

Printed circuit board 260 can include reference sensor 237, which can be loaded similarly to sensors of sensor arrays 230 under a no-load condition, as described more particularly below. Reference sensor 237 is used for test and calibration of sensor arrays 230. This provides substantial benefits in both the assembly and cost of measurement device 4300. In a further embodiment, sensors of sensor array 230 and reference sensor 237 are integrated into printed circuit board 260 and more specifically in respective sections 236, with sensors of sensor array 230 forming sensor arrays 230.

In some aspects, sensor 237 is located more centrally inward along inner edge 236b of respective section 236, opposite curved outer edge 236b of each peripheral section of section 236. At least one reference sensor 237 can be positioned on each side of printed circuit board 260 within array 230. For example, sensors 237 can be positioned on an edge between vertices of the triangular shaped area and between sensors of sensor array 230 on those vertices. In one embodiment, reference sensor 237 is formed identical to sensors of sensor array 230.

Traces 239 can extend from printed circuit board 260 in or on section 236 to electrically connect with sensors of sensor array 230 and reference sensor, 237. Traces 239 in some aspects can include one or more coils 232, or dense portions, between printed circuit board 260 and corresponding sensor 237 and/or sensors of sensor array 230. In some aspects, the one or more coils 232 are advantageous to ensure that the base capacitance of all sensors is as close as possible. In some aspects, the base capacitance can vary based on trace capacitance but can be approximately 16.4 pF, though other values are contemplated as needed or required. Printed circuit board 260 can be a flexible unitary substrate or circuit board. In other words, sections of printed circuit board 260 can be formed as a single structure. In some aspects, traces 239 may be the same length, so base capacitance can be substantially the same for each sensor of sensor array 230 and each reference sensor 237. In some aspects, traces 239 be one or more dense portions of printed circuit board 260, and the dense portions may be selectively positioned relative to other components of printed circuit board 260 to avoid trace 239 over-heating. For example, dense portions of traces 239 may be spaced from each other to avoid over-heating of portions of the printed circuit board 260.

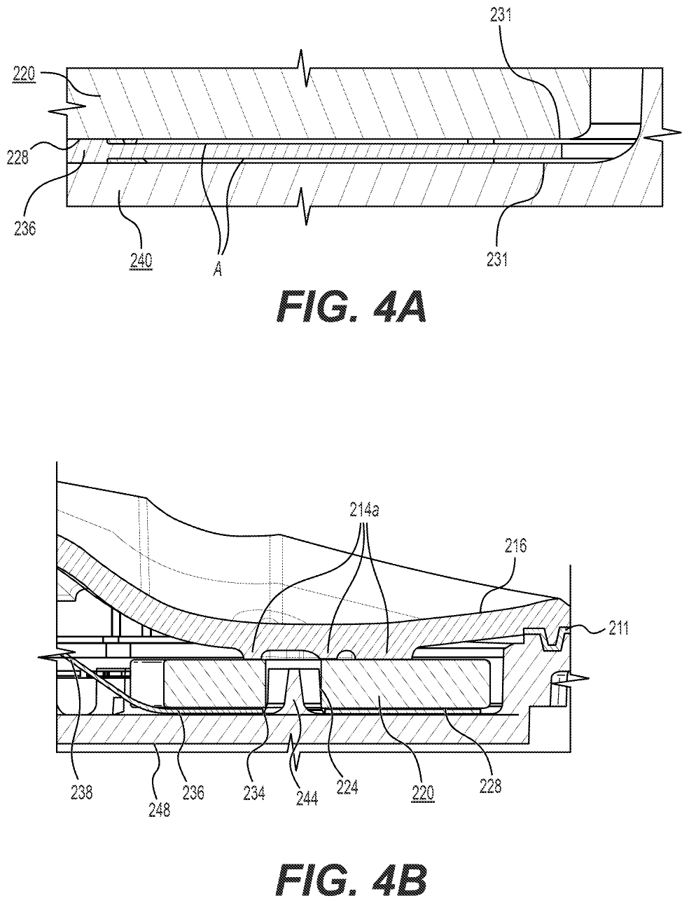

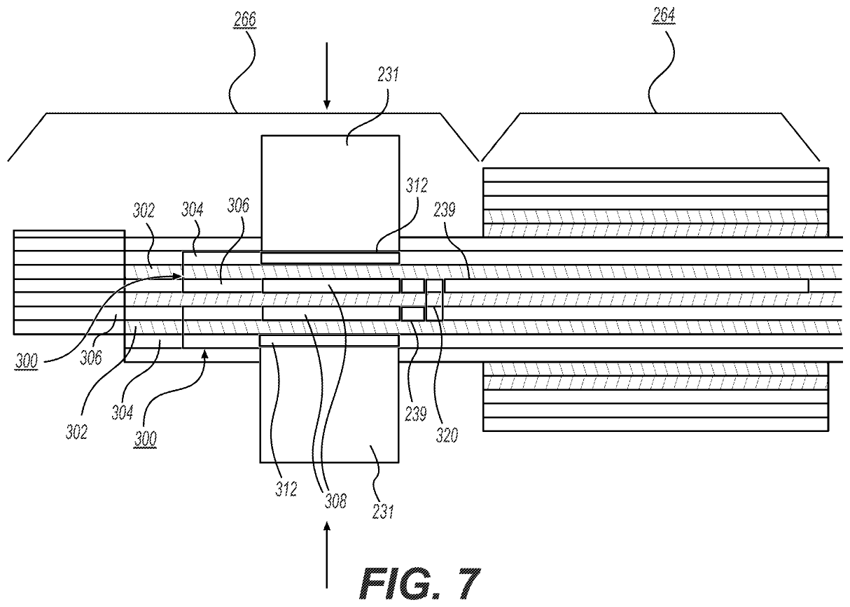

Referring now to how the load path is deflected around sensor 237, FIG. 4A, is a close-up cross-sectional view taken from section 4A-4A in FIG. 3B showing structure 240, section 236 of printed board 260, and pads 231 at the posterior-most sensor of sensor array 230. An air gap A may be created by load pads 231, and air gap A may be positioned between section 236 of printed board 260 and structure 240 and/or between section 236 and plate 220. In some aspects, this load path advantageously avoids reference sensor 237 as a result of load pads 231 being etched down so that between pads 231 and respective plate 220, air gap A is created, as shown in FIG. 4A. In some aspects, load pads 231 being etched down, load pads 231 can protrude from a respective section 236 to form air gap A so as to allow space between reference sensor 237 and load plate 220. In general, load pads 231 can include a non-compressible material such as metal, composite material, or a polymer as well as a metal such as copper or gold. Load pads 231 can couple to a terminal or electrode of the corresponding sensor, such as sensors of sensor array 230. Load pads 231 can couple to a capacitor plate of the capacitive sensor, as shown and described more clearly in FIG. 7. Load pads 231 can be coupled to shields that couple to ground for preventing parasitic coupling to the sensors. In some aspects, printed circuit board 260 can include one or more raised columns to align with sensors of sensor array 230 whereas no such column can be present for reference sensor 237. Raised columns may reduce load applied to reference sensor 237 while concentrating load application onto sensor array 230.

In some aspects, air gap A can be created at least in part because pads 231 are etched down into the substrate of section 236 as well as the one or more corresponding raise columns. The cross-sectional views of FIG. 4A shows air gap A above and below section 236. In some aspects, air gap A is advantageous to minimize load paths through sensor 237 and some instances, ensure that there are no possible load paths through sensor 237. In some aspects, reference sensor 237 is structurally similar to sensors of sensor array 230 except for its top and bottom layers of the etched down copper that create the load pads 231 (e.g., buttons) the load plates 220 rest on. In some aspects, an acid is used to remove copper of printed circuit board 260 in areas where it is not needed in the etching process of load pads 231. A mask can be applied to the areas of printed circuit board 260 where it is not desirous to remove any copper.

For example, reference sensor 237 may not include load pads 231, which may provide a space between load plates 220 and reference sensor 237 and avoid loading of reference sensor 237. In some aspects, vias (e.g., via bridge 320 of FIG. 7) at or adjacent each sensor of sensor array 230 will also experience little to no loading as a result of the discussed air gaps A.

Referring to support structure 240 of FIG. 2C and more particularly illustrated in FIGS. 3A and 3B, printed circuit board 260 is shown assembled with load pads 220 within support structure 240. Support structure 240 can include one or more cavities 246 that underlie articular surfaces 216. Support structure 240 can include one or more alignment features 249 for retaining and aligning printed circuit board 260 as well as corresponding circuitry 250. In the example, electronic circuitry 250 associated with printed circuit board 260 can be placed in a region of support structure 240 defined by or in communication with one or more alignment features 249, which in some aspects, can support placement of some or all of printed circuit board 260 and/or circuitry 250 therein. In the example of FIG. 2B, alignment feature 249 can retain and align support structures 240 and 210 to each other. Support structure 210 can also include alignment structure 212 that couple to alignment feature 249. Support structure 240 further includes the peripheral receiving channel of flange 242 that mates with flange 211 to support forming the hermetic seal that isolates electronic circuitry 250 and other components of printed circuit board 260 (e.g., sensors 237, sensor arrays 230, etc.). In some aspects, structure 240 can include a sturdy floor that reflects the reaction force from the sensors of sensor array 230 without much, if any, deflection during loading. In some aspects, the same is true when any shims are attached to it, which provides consistent loading characteristics.

Similarly, alignment structure 244 can extend upward from a lower surface 247 of structure 240 to retain and align load plates 220 and sensor arrays 230 to support structure 240. In some aspects, alignment structure 244 can be an alignment pin, dowel or other extrusion that extends from the inner surface of structure 240. In some aspects, alignment structure 244 can precisely align sensors of sensor array 230 and reference sensor 237 of sensor array 230 to articular surfaces 216 of support structure 210 for determining position of applied force, pressure, of load to measurement device 200.

Referring back to FIGS. 2C and 3A, certain aspects of example load plates 220 are shown. Load plate 220 can include an upper surface 226, a lower surface 228, and a plurality of openings 224 for aligning and positioning to alignment structure 244 of support structure 240. In an assembled state, a force, pressure, or load applied to articular surfaces 218 of structure 210 can be transferred to load plates 220 so as to distribute the load to the sensors of sensor array 230. In one embodiment, load plates 220 can include a rigid structure that support and distribute loading over a large surface area. Load plates 220 can include a metal or polymer material. In the example, load plates 220 includes stainless steel.

As shown in FIG. 2C, device 200 is shown in its exploded state with load plates 220, printed circuit board 260, electronic circuitry 250, load pads 231 proximate sensors of sensor arrays 230. A pair of load plates 220 are shown positioned below area 214 so as to ensure that all the sensors of array 230 are loaded parallel to the plane in which the sensor lie. In turn, an X-Y translation of structure 210 is all but eliminated and converting to a single direction -Z force for printed circuit board 260 therebelow. Load plates 220 in some aspects can also prevents edge loading to the sensors of array 230 by distributing the load over the entire sensor thereby avoiding additional electrical noise to a capacitor that is, for example, showing more compression on one side.

Electronic circuitry 250 is more particularly shown in FIGS. 3A, 3B, 6A, and 6B. In FIG. 3B with load plate 220 and structure 210 removed for viewing purposes only, electronic circuitry 250 is shown coupled to arrays 230 so as to electronically communicate with arrays 230. Electronic circuitry 250 can include a power source such as one or more batteries (e.g., one or more batteries 262), capacitor, or inductor that can power the device while measurements are taken. Electronic circuitry 250 can include a transceiver and antenna for wireless communication to a remote system, such as receiving station 110. In one embodiment, communication is short range, typically less than 10 meters.

Electronic circuitry 250 and sensor arrays 230 can be housed in measurement device 200 in the enclosure between structures 210, 240 under approximately no load or lightly loaded when support structures 210, 240 are coupled together. In some aspects, sensors of sensors of sensor array 230, as well as reference sensors 237, can be elastically compressible capacitors. The capacitors of sensors of sensor array 230, 237 can be configured to generate a signal having a time period corresponding to the force, pressure, or load. The signal from sensors of sensor array 230 and/or reference sensor 237 can be received by electronic circuitry 250. The measurements can be stored in memory on measurement device 200. Alternatively, measurement data can be transmitted to a remote system. The measurement data in either instance can be used to calculate the force, pressure, or load magnitudes as well as the position of the force, pressure, or load on the articular surface 218.

Aspects of sensor array 230 are more particularly shown and described in FIGS. 2C, 3B, 4A, 4B, and 6A-6B. In some aspects, sensors of sensor array 230 can be placed between load bearing surfaces for measurement. Sensors of sensor array 230 can be used to determine force, pressure, or load magnitude and the position of force, pressure, or load applied to the surface of measurement device 200. As shown in FIG. 2C, each sensor array 230 can include three sensors in the vertices of a triangular shaped area corresponding to lateral sections 236 of printed circuit board 260. In some aspects, the vertices of sensor array 230 can align with vertices 214b shown more clearly in FIG. 5C. Sensors of sensor array 230 can underlie load pads 231. Load pads 231 are shown more particularly in FIG. 4A, which is a close-up cross sectional view of section 4A-4A of FIG. 3B. Pads 231 can support load plates 220 for directing a force, pressure, or load applied to articular surfaces 218 to sensor array 230. The stiffness of plate 220 may prevent the deflection at high loads to areas, such as air gap A, that are not the sensors. When the sensors are arranged in array 230 and the contact between all the components as shown in FIG. 4A in its preloaded state, this will prevent plate 220 from “tipping” or edge loading any of the sensors because of the prevented rotation.

Referring to FIG. 4B, a side cross section view of section 4B-4B from FIG. 3B is provided. Here, it can be seen that ribbed protrusions 214a of area 214 are in contact with plate 220 to ensure that the loads applied to the condyles of articular surfaces 216 are transmitted to the centralmost point of area 214. In some aspects, a consular bias can be applied to surfaces 216 to facilitate loading through the central most point of area 214. The consular bias can allow for the loads that are applied too mesial in either condyle to be pushed towards the lowest spot on the condyle, which advantageously correlates to a more centralized load in area 214. In some aspects, the geometries of surface 216 can come within +/−0.5 mm of the shape of the tibial prosthetic component 106.

In some aspects, device 200 may only include one reference sensor 237 per section 236 in order to give a baseline force value for the condyle associated with respective section 236. In some aspects, reference sensor 237 can be located as close to as many load sensors of sensor array 230 as possible without absorbing any energy from the principal load path. This allows for reference sensor 237 to be in the same exact environment as the sensors of sensor array 230 measuring load, and may provide more accurate calibration of sensor array 230 compared to positioning reference sensor 237 farther from sensor array 230. Reference sensor 237 can endure similar environmental conditions (e.g., similar temperature, humidity, etc.) as sensors of sensor array 230 without getting loaded as a result of the femur implant/trial load path. In some aspects, reference sensor 237 can be by calibrating each sensor array 230 on in each lateral section 236 based on separate reference sensor 237 information (e.g. a first reference sensor 237 on a first lateral side and a second reference sensor 237 on a second lateral side

In some examples, some or all of sensors of sensor array 230 can be integrally formed in printed circuit board 260. The sensors of sensor array 230 can be placed at the vertices of a polygon (e.g., vertices 214b of area 214) and include one or more reference sensors 237 positioned along an inner edge (e.g., adjacent edge 236b) of the formed polygon. In the example, the polygon is a triangle that corresponds to area 214. The area of the polygon relates or corresponds to area 214 which underlies articular surface 216 of support structure 210. Sensors of sensor array 230 can measure the force, pressure, or load, and the position of the force, pressure, or load in at least the area of the polygon and the corresponding area of articular surfaces 216.

Aspects of sensor array 230 are further described in FIGS. 6A and 6B. In some aspects, sensors of sensor array 230 and reference sensor 237 are shown integrated into printed circuit board 260 (e.g., via section 236) and more specifically in the peripheral sections of sensor array 230. The sensors of sensor array 230 and reference sensor 237 can be elastically compressible capacitors that are formed in section 236 of array 230. Reference sensor 237, in some aspects, can be the same size as those of sensor array 230 and measure the capacitance with no load, to account for temperature and atmospheric pressure variances. As an alternative example, a piezo-resistive sensor can be formed within the printed circuit board 260, including section 236, by screen printing or using photolithographic techniques to create regions where the compressible conductive material is deposited.

Referring now to FIG. 7, example capacitors 300 contemplated for use with printed circuit board 260 are shown, including capacitors contemplated for use with sensors of sensor array 230 and/or sensor 237. In the example, capacitors 300 are shown positioned in the flexible portion 266 of printed circuit board 260 adjacent rigid portion 264 of printed circuit board 260. The example capacitors 300 can be used as a force, pressure, or load sensor for the muscular-skeletal system. In some aspects, capacitors 300 can include multiple interconnected alternating conductive and dielectric layers. The conductive layers can be patterned to form conductive regions and interconnect. Applying a force, pressure, or load to such a multi-layer is advantageous since its dielectric layers can deform and small deformations of its dielectric layers can rebound elastically when stimulus is removed. Deformation of the dielectric layer in turn changes the dielectric thickness of capacitor 300 and its capacitance value.

In some aspects, capacitor 300 can be used to produce an electrical signal to receive, analyze, and provide the measurement data for sensors of sensor array 230 and/or reference sensor 237. In some aspects, capacitor 300 can include a dielectric layer 302, a dielectric layer 304, and a dielectric layer 306. Material contemplated for use with layers 302, 034, 306 can include at least one of polymer, polyester, an aramid, an adhesive, silicon, glass, and composite material. Capacitor 300 as shown can include more than two capacitors in series mechanically.

In the depicted example of FIG. 7, two capacitors 300 can be connected in mechanical series. However, fewer or greater number of capacitors 300 are contemplated as needed or required and capacitors 300 can also be in parallel. A conductive region 308 can be included with a predetermined area (e.g., a 4.2 mm disk). The predetermined area, layer thickness, and dielectric constant of dielectric layer 302 can be used to determine the capacitance of capacitor 300. In one embodiment, conductive region 308 overlies, has substantially equal area, and is aligned to another conductive region 308 therebelow. The illustrated regions 308 can be coupled in common via bridge 320. A conductive region 312 can be positioned overlying the uppermost capacitor 300 of FIG. 7.

In some aspects, conductive regions 312 can be coupled to ground forming a shield. Region 312 can be in communication with layer 304. Similarly, the lowermost capacitor 300 of FIG. 7 can include underlying region 312, including layer 302 and in communication with layer 304. Region 312 can have approximately equal area with and be aligned to conductive region 308. Previously discussed load pad 231 can overly conductive region 312. Load pad 231 can prevent damage to region 312 due to a force, pressure or load applied thereto. Capacitor 300, when used in sensors of this disclosure including reference sensor 237 or those of sensor array 230, can be coupled to electronic circuitry 250 via traces 239 travelling between the flexible portion 266 and the rigid portion 264. In the example of FIG. 7, a force, pressure, or load is applied by the muscular-skeletal system to load pads 231. The force, pressure, or load compresses corresponding capacitors 300. Dielectric layers 302, 304, and 306 can compress under the force, pressure, or load.

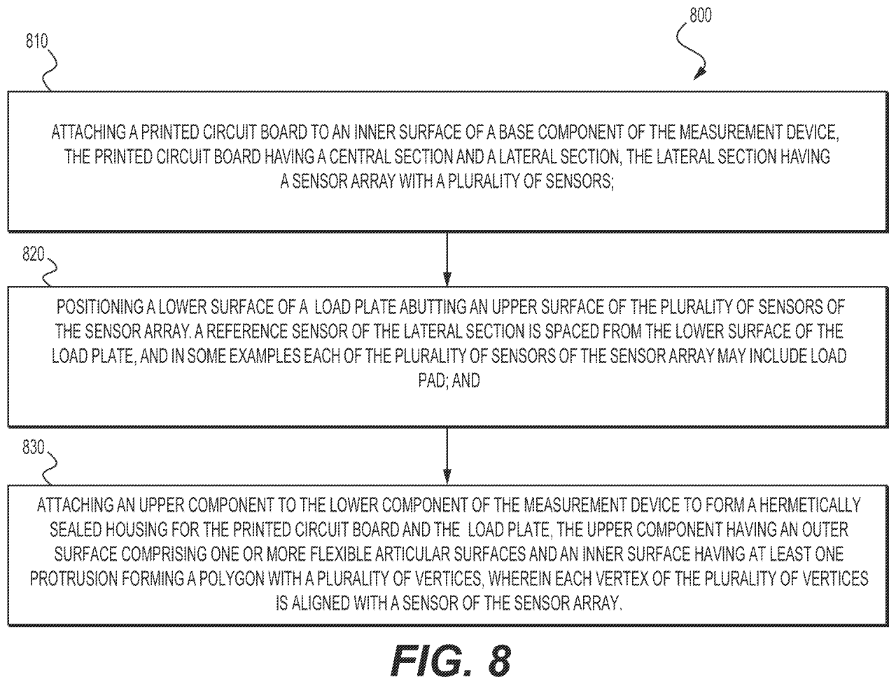

FIG. 8 depicts a flow diagram of a method 800 of assembling a measurement device according to certain aspects of this disclosure. Step 810 of method 800 can include attaching a printed circuit board to an inner surface of a base component of the measurement device, the printed circuit board having a central section and a lateral section, the lateral section having a sensor array with a plurality of sensors. Step 820 of method 800 can include positioning a lower surface of a rigid load plate abutting an upper surface of load pads associated with the plurality of sensors of the sensor array, each load pad being etched down and in contact with the lower surface of the rigid load plate so that a reference sensor of the lateral section is positioned within an air gap formed between the lower surface of the rigid load plate and an upper surface of the load pad. Step 830 of method 800 can include attaching an upper component to the lower component of the measurement device to form a hermetically sealed housing for the printed circuit board and the rigid load plates, the upper component having an outer surface having one or more flexible articular surfaces and an inner surface having a pair of areas each having inwardly oriented protrusions aligned with the vertices of the polygon associated with the sensor array. In some aspects, method 800 can end after some or all of these steps. In other aspects, additional steps according to the examples described herein can be performed.

The present disclosure is applicable to a wide range of medical and nonmedical applications including, but not limited to, frequency compensation; control of, or alarms for, physical systems; or monitoring or measuring physical parameters of interest. The level of accuracy and repeatability attainable in a highly compact sensing module or device may be applicable to many medical applications monitoring or measuring physiological parameters throughout the human body including, not limited to, bone density, movement, viscosity, and pressure of various fluids, localized temperature, etc. with applications in the vascular, lymph, respiratory, digestive system, muscles, bones, and joints, other soft tissue areas, and interstitial fluids.

While the present disclosure has been described with reference to particular embodiments, those skilled in the art will recognize that many changes may be made thereto without departing from the spirit and scope of the present disclosure. Each of these embodiments and obvious variations thereof is contemplated as falling within the spirit and scope of the disclosure.

What is claimed is:

1. A measurement device for measuring force, pressure, or load applied by a musculoskeletal system, comprising:
a first component comprising:
an outer surface comprising one or more flexible articular surfaces, and
an inner surface comprising a first area comprising protrusions defining a first polygon with a plurality of vertices;
a first load plate in contact with the first area;
a printed circuit board comprising a central section and a first lateral section, the first lateral section comprising:
a first sensor array comprising a plurality of first sensors, each sensor of the first sensor array positioned in alignment with a vertex of the first polygon;
a first reference sensor spaced from the first load plate such that a load applied to the outer surface of the first component compresses at least one of the plurality of first sensors while the first reference sensor remains unloaded;
electronic circuitry configured to control a measurement process, receive measurement data from the first sensor array, and transmit measurement data; and
a second component configured to attach to the first component to form a hermetically sealed housing for the printed circuit board.

2. The device of claim 1, wherein at least two of the plurality of first sensors are positioned in alignment with opposite vertices of an inner lateral edge of the first polygon,
wherein the first lateral section comprises a curved outer edge opposite a substantially straight inner edge,
the first reference sensor of the first lateral section being positioned adjacent the inner edge and between the at least two of the plurality of first sensors positioned in alignment with opposite vertices of the inner lateral edge.

3. The device of claim 1, further comprising:
a load pad in contact with a lower surface of the first load plate and coupled to a sensor of the first sensor array; and
an air gap positioned between the lower surface of the first load plate and the first reference sensor.

4. The device of claim 1, wherein the first lateral section of the printed circuit board comprises one or more raised columns, wherein each of the one or more raised columns is aligned with a sensor of the first sensor array.

5. The device of claim 1, wherein the inner surface comprises a second area comprising a second polygon with a plurality of vertices; the device further comprising:
a second rigid load plate positioned in contact with the second area;
wherein the printed circuit board comprises:
a second lateral section positioned on an opposite side of the central section from the first lateral section, and
a second sensor array comprising a plurality of second sensors, wherein each sensor of the plurality of second sensors is positioned in alignment with a vertex of the second polygon of the second area; and
wherein a second reference sensor is spaced from a lower surface of the second rigid load plate such that a load applied to the outer surface of the first component compresses at least one of the plurality of second sensors while the second reference sensor remains unloaded.

6. The device of claim 1, further comprising a plurality of traces of the printed circuit board that electrically couple the first reference sensor and each of the plurality of first sensors to the central section of the printed circuit board, wherein each trace of the plurality of traces comprises at least one coil or dense section.

7. The device of claim 6, wherein each trace of the plurality of traces is approximately the same length.

8. The device of claim 7, wherein each coil or dense section is spaced from each other coil or dense section.

9. The device of claim 1, wherein the first reference sensor is configured to be used for calibration.

10. The device of claim 1, wherein each of the first sensors comprise a capacitor including an elastic dielectric.

11. The device of claim 10, wherein each of the capacitors of the first sensors include a plurality of capacitors.

12. A measurement device for measuring force, pressure, or load applied by a musculoskeletal system, comprising:
a first component comprising an outer surface comprising one or more articular surfaces and an inner surface comprising a first area comprising a protrusion defining a first polygon;
a load plate in contact with the protrusion;
a printed circuit board comprising a central section and a first lateral section extended from the central section, the first lateral section comprising:
a first sensor array comprising a plurality of first sensors, wherein each of the first sensors is in contact with a lower surface of the load plate, the first sensor array comprising:
a first sensor positioned in a central portion of a periphery of the first lateral section,
a second sensor in a first corner of the periphery, and
a third sensor in a second corner opposite the first corner of periphery, wherein each of the first sensor, the second sensor, and the third sensor is aligned with a vertex of the first polygon;
a first reference sensor positioned between the second sensor and the third sensor, wherein the first reference sensor is spaced from the load plate; and
electronic circuitry configured to control a measurement process, receive measurement data, and transmit data from the plurality of first sensors and the first reference sensor; and
a second component configured to attach to the first component to form a housing for the printed circuit board.

13. The device of claim 12, wherein each of the first sensors includes a capacitor.

14. The device of claim 13, wherein each of the first sensors includes a plurality of capacitors.

15. The device of claim 12, wherein the first polygon is triangular shaped with an inner lateral edge aligned parallel with an inner lateral edge of the load plate, and at least two of the plurality of first sensors are positioned on opposite vertices of the inner lateral edge.

16. The device of claim 12, wherein the first reference sensor is positioned between two of the first sensors.

17. A method of assembling a measurement device for measuring force, pressure, or load applied by a musculoskeletal system, comprising:
attaching a printed circuit board to an inner surface of a base component of the measurement device, the printed circuit board comprising a central section and a lateral section, the lateral section comprising a sensor array comprising a plurality of sensors;
positioning a lower surface of a load plate abutting each of the plurality of sensors of the sensor array, wherein a reference sensor is spaced from the load plate; and
attaching an upper component to the lower component of the measurement device to form a hermetically sealed housing for the printed circuit board and the load plate, the upper component comprising an outer surface including one or more flexible articular surfaces and an inner surface comprising at least one protrusion forming a polygon with a plurality of vertices, wherein each vertex of the plurality of vertices is aligned with a sensor of the sensor array.

18. The method of claim 17 Error! Reference source not found, further comprising:
applying a load to the outer surface of the upper component to compress at least one of the plurality of sensors while the reference sensor remains unloaded.

19. The method of claim 17, further comprising:
positioning on or more raised columns of the printed circuit board in alignment with each of the plurality of sensors of the sensor array.

20. The method of claim 17, further comprising:
electrically coupling the central section of the printed circuit board to the reference sensor and each of the plurality of sensors of the sensor array with a corresponding trace having at least one coil.
